(19) United States
(12) Patent Application Publication
(10) Pub. No.: US 2024/0335180 A1
(43) Pub. Date: Oct. 10, 2024

(54) METHODS AND SYSTEMS FOR A MODIFIED DEMATCHING LAYER

(71) Applicant: GE Precision Healthcare LLC, Wauwatosa, WI (US)

(72) Inventor: Jeremie Barrel, Opio (FR)

(21) Appl. No.: 18/295,789

(22) Filed: Apr. 4, 2023

(51) Int. Cl. A61B 8/00

(52) U.S. Cl. CPC A61B 8/4483; A61B 8/4444

(57) ABSTRACT

Various methods and systems are provided for a modified dematching layer for a transducer. In one example, the dematching layer includes a matrix having a first acoustic impedance and inclusions embedded in the matrix. The inclusions may have a second acoustic impedance that is lower than the first acoustic impedance and may be configured to attenuate acoustic energy at an acoustic band gap of the modified dematching layer.

### FIELD

Embodiments of the subject matter disclosed herein relate to a transducer for a medical device.

### BACKGROUND

Transducers are used in a variety of applications to convert energy from a physical form to an electrical form. For example, a transducer may include a piezoelectric material which generates electrical voltage from a mechanical stress or strain exerted on the material and converts electrical signals into mechanical vibrations. Transducers configured to operate in a quarter-wavelength vibration mode may demonstrate enhanced sensitivity and bandwidth relative to conventional half-wavelength vibration mode piezoelectric resonators. Further, the thinner quarter-wavelength vibration resonance piezoelectric materials, relative to piezoelectric materials providing half-wavelength vibration resonance, may allow an electrical impedance of the transducer to match that of a corresponding imaging system more closely.

Both quarter-wavelength and half-wavelength vibration mode transducers may include a backing layer located behind the piezoelectric material, relative to a direction of signal propagation. The backing layer may be formed of a material with an acoustic impedance similar to that of the piezoelectric materials to provide effective damping in the transducer. For transducers configured to operate in the quarter-wavelength vibration mode, a dematching layer is included, thereby demanding incorporation of the thinner piezoelectric material. The dematching layer allows the thinner piezoelectric material to achieve a same target resonant frequency as a thicker piezoelectric material by modifying a mode of resonance of the piezoelectric material. The dematching layer is positioned behind the piezoelectric material, which may decrease insertion losses at the transducer while increasing the transducer bandwidth.

A footprint of the transducer, relative to the direction of signal propagation, is therefore dependent on a thickness of an acoustic stack of the transducer, where the acoustic stack includes the piezoelectric material, the backing layer, the dematching layer, as well as other acoustic layers. For example, the other acoustic layers may include at least one electrical circuit that electrically couples the acoustic stack to a voltage source, thereby enabling application of a potential to the piezoelectric material to induce vibration thereat. The electrical circuit may be a flex circuit or an application-specific integrated circuit (ASIC). When the ASIC is used, the ASIC may be positioned directly behind and in contact with the piezoelectric material. The backing layer may be located between the ASIC and the dematching layer. As the material of the backing layer may be a non-conductive material, formation of electrical connections through the backing layer may be demanded to provide electrical continuity between the ASIC and the dematching layer.

### BRIEF DESCRIPTION

In one embodiment, a modified dematching layer for a transducer includes a matrix having a first acoustic impedance and inclusions embedded in the matrix, the inclusions having a second acoustic impedance lower than the first acoustic impedance and configured to attenuate acoustic energy at an acoustic band gap of the modified dematching layer. In this way, a thickness of the transducer may be reduced, thereby decreasing its footprint, while increasing a sensitivity of the transducer.

In another embodiment, a method for fabricating a transducer includes fabricating a metamaterial with an acoustic band gap corresponding to a resonance frequency of a piezoelectric material. The metamaterial may have a matrix with a first acoustic impedance and inclusions with a second acoustic impedance, the second acoustic impedance lower than the first acoustic impedance. The metamaterial may be incorporated into an acoustic stack, where the metamaterial is both a dematching layer and a backing of the acoustic stack.

In yet another embodiment, an acoustic stack includes a piezoelectric material and an electrical circuit in contact with the piezoelectric material. The acoustic stack further includes a modified dematching layer positioned adjacent to the piezoelectric material with the electrical circuit positioned therebetween. The modified dematching layer may be formed of a metamaterial having a matrix with a first, higher acoustic impedance and inclusions with a second, lower acoustic impedance. The inclusions may be distributed within the matrix to attenuate acoustic energy at a resonance frequency of the piezoelectric material.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1A shows a first example of an acoustic stack of an ultrasound transducer having a modified dematching layer, according to an embodiment;

FIG. 1B shows a second example of an acoustic stack of an ultrasound transducer having a modified dematching layer, according to an embodiment;

FIG. 2 shows a schematic illustration of stray acoustic energy propagation through a conventional dematching layer, according to an embodiment;

FIG. 3 shows a schematic illustration of stray acoustic energy propagation through a modified dematching layer, according to an embodiment;

FIG. 4 shows graph of acoustic energy attenuation at a band gap of a modified dematching layer, according to an embodiment;

FIG. 5 shows a first example of inclusions of a modified dematching layer, according to an embodiment;

FIG. 6 shows a second example of inclusions of a modified dematching layer, according to an embodiment;

FIG. 7 shows a third example of inclusions of a modified dematching layer, according to an embodiment;

FIG. 8 shows a fourth example of inclusions of a modified dematching layer, according to an embodiment;

FIG. 9 shows a fifth example of inclusions of a modified dematching layer, according to an embodiment; and

FIG. 10 shows an example of a method for fabricating an acoustic stack with a modified dematching layer, according to an embodiment.

### DETAILED DESCRIPTION

The following description relates to various embodiments of a transducer for a medical device. The transducer may include an acoustic stack formed of various layers, including a piezoelectric material, a matching, a backing, and an electrical circuit. By configuring the piezoelectric material with a thickness to provide a quarter-wavelength vibration frequency, a bandwidth and sensitivity of the transducer may be increased relative to a piezoelectric material with a half-wavelength vibration frequency.

As a resonance frequency of a piezoelectric material is dependent on its thickness, the quarter-wavelength vibration frequency piezoelectric material is thinner than the half-wavelength vibration frequency piezoelectric material, which allows the piezoelectric material to vibrate at a higher frequency. A dematching layer is also included in an acoustic stack of the transducer when the piezoelectric material has the quarter-wavelength vibration frequency. A bandwidth of the transducer may vary according to a thickness of the dematching layer. The dematching layer may have a higher acoustic impedance than the piezoelectric material and may allow the transducer, when operating in a quarter-wavelength vibration mode (e.g., a ¼-λ transducer), to generate acoustic signals with a resonance frequency corresponding to that achieved by a transducer operating in a half-wavelength vibration mode (e.g., a ½-λ transducer) without a dematching layer. For example, a ratio of the acoustic impedance of the dematching to the acoustic impedance of the piezoelectric material may be at least 2. As an example, the piezoelectric material may be lead zirconate titanate (PZT) with an acoustic impedance of approximately 35 megaRayls (MR) while the dematching layer may be cobalt-bonded tungsten carbide (WC) having an acoustic impedance of approximately 100 MR. The ¼-λ transducer may therefore be used for the same applications as the ½-λ transducer but may provide, for example, higher imaging quality while reducing a footprint of the acoustic stack.

Maintaining a thickness of the acoustic stack as small as possible may be desirable, particularly for applications, such as intravenous insertion, where transducers with small dimensions are demanded. However, reduction of the footprint of the acoustic stack may be hindered by a demand for incorporation of multiple layers, e.g., one or more matching layers, the piezoelectric material, the dematching layer, the backing layer, etc., in order to achieve a desired performance of a transducer. In particular, the backing layer may be a thickest component of the acoustic stack.

Furthermore, when the dematching layer is included, the dematching layer may be arranged behind the piezoelectric material, relative to a direction of signal propagation, in direct contact with the piezoelectric material. As such, in examples where at least one application-specific integrated circuit (ASIC) is included in the acoustic stack as an electrical circuit coupled to the piezoelectric material, the backing may be arranged between the ASIC and the dematching layer, e.g., behind the dematching layer and in front of the ASIC. In order to provide electrical continuity between the ASIC and the dematching layer, formation of electrical connections through the backing may be required, which may complicate manufacturing and increase costs.

In one example, the issues described above may be addressed by configuring an acoustic stack of a transducer with a dematching layer formed of a metamaterial that provides acoustic effects of both a backing and a dematching layer. By forming the dematching layer from the metamaterial, the dematching layer may be engineered with specific, desired properties. This may allow the dematching layer to be readily customized to various applications without demanding extensive development and processing strategies.

The metamaterial may be composed of a variety of materials and configured to have specific optical and electromagnetic characteristics. As an example, the metamaterial may be artificially modified, relative to a naturally occurring parent material, at a nanoscale basis to behave differently from the parent material. The properties of the metamaterial may arise primarily from its structure, rather than its composition. Unlike conventional composite materials, the metamaterial may demonstrate a band gap, e.g., an acoustic band gap, which may be varied according to the structure of the metamaterial. In this way, the metamaterial may be engineered to have a specific attenuating frequency to enable modification of acoustic energy passing therethrough. Metamaterials may be fabricated via various techniques, including electron-/ion-beam lithography, photolithography, interference lithography, shadow lithography, nanoimprint lithography, as well as by 3D printing methods, such as direct laser writing, and electrochemical deposition. The metamaterial may therefore be manufactured via processes selected according to desired costs and time.

The metamaterial may have a matrix formed of a first, higher acoustic impedance material, e.g., having a higher acoustic impedance than a piezoelectric material of the acoustic stack, with embedded inclusions having a second, lower acoustic impedance, e.g., an acoustic impedance lower than the piezoelectric material. Further, the embedded inclusions may demonstrate high acoustic attenuating properties. The matrix may therefore operate as a dematching layer of the acoustic stack while the embedded inclusions have an effect analogous to a backing. The inclusions may be incorporated at a ratio relative to the matrix such that the acoustic impedance of the matrix is not reduced by the presence of the inclusions, which may be achieved by maintaining an acoustic impedance ratio of the metamaterial to the piezoelectric material of at least 2. Further, the metamaterial may have an acoustic band gap corresponding to a resonance frequency of the piezoelectric material, in contrast to a composite material, which may not exhibit an acoustic band gap.

By incorporating properties of both the dematching layer and the backing, the resulting modified dematching layer may have increased acoustic absorbance relative to a conventional dematching layer, and enhance a sensitivity and bandwidth of the transducer. The modified dematching layer may also offset interferences from resonance frequencies generated at other acoustic stack components. As an example, in some instances, such as when the electrical circuit of the acoustic stack is configured as a flex circuit, the flex circuit may produce resonances in response to acoustic energy generated by the piezoelectric material.

For example, although an interface between a dematching layer and a piezoelectric material may be configured to direct most of the acoustic energy generated at the piezoelectric material to a rear of the transducer, some of the acoustic energy may nonetheless reach the flex circuit. The acoustic energy that reaches the flex circuit may cause the flex circuit to resonate. The resonances of the flex circuit may occur at frequencies falling within a frequency spectrum of the transducer, which may lead to generation of artifacts and may degrade a performance of the transducer. To mitigate this, properties of the modified dematching layer may be optimized to target a resonance frequency of the flex circuit (which may also be the resonance frequency range of the piezoelectric material). The modified dematching layer may thereby absorb undesired acoustic energy generated by the flex circuit.

Alternatively, when the electrical circuit is the ASIC, the modified dematching layer may be directly coupled to the ASIC, precluding reliance on a backing layer with electrical connections therethrough to electrically connect the ASIC to a conventional dematching layer. Thus, electrical losses may be decreased and manufacturing of the backing may be simplified. Further, incorporation of the modified dematching layer may allow the backing layer thickness to be decreased, thereby reducing a bulkiness of the backing layer. In yet other examples, the backing may be obviated and omitted from the acoustic stack. By configuring the transducer with the modified dematching layer, the transducer may have a smaller footprint and demonstrate enhanced sensitivity and bandwidth without increasing manufacturing costs. The smaller footprint of the transducer may expand a use of the transducer for different applications and minimize discomfort to a patient.

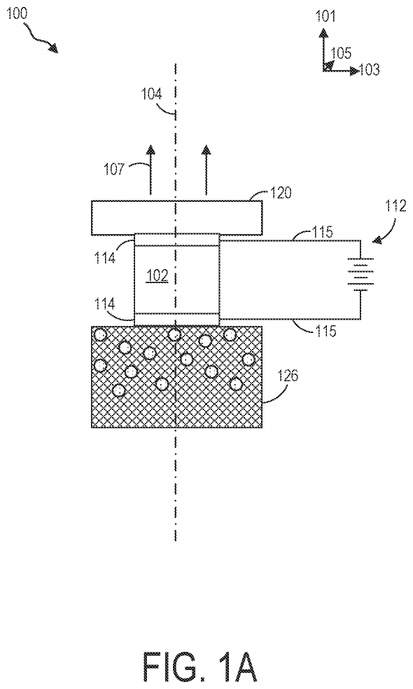

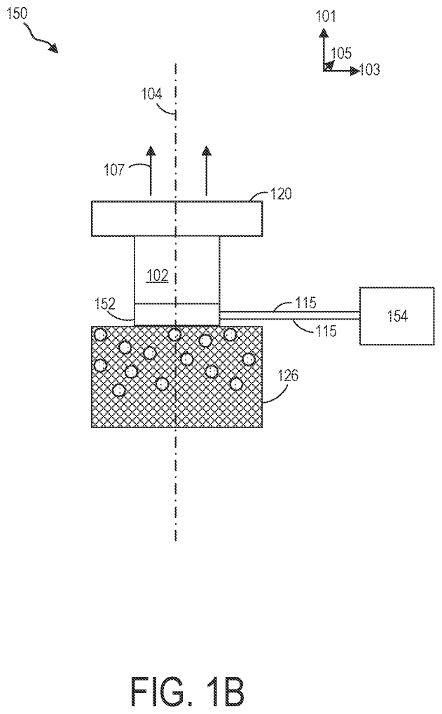

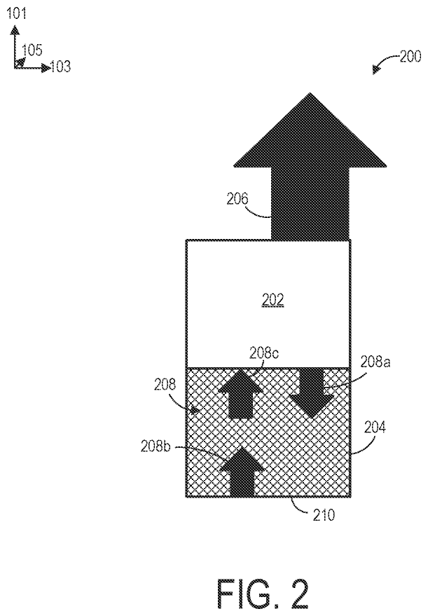

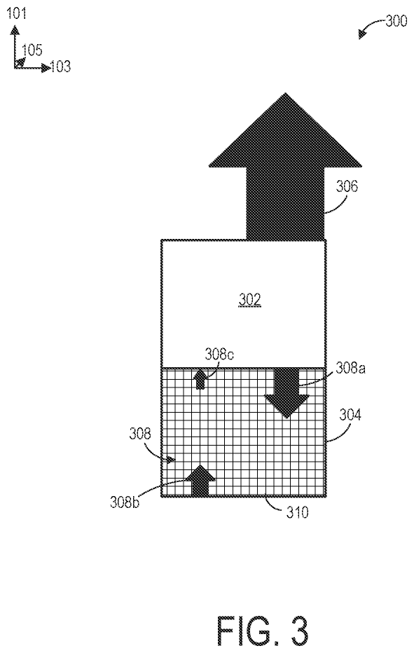

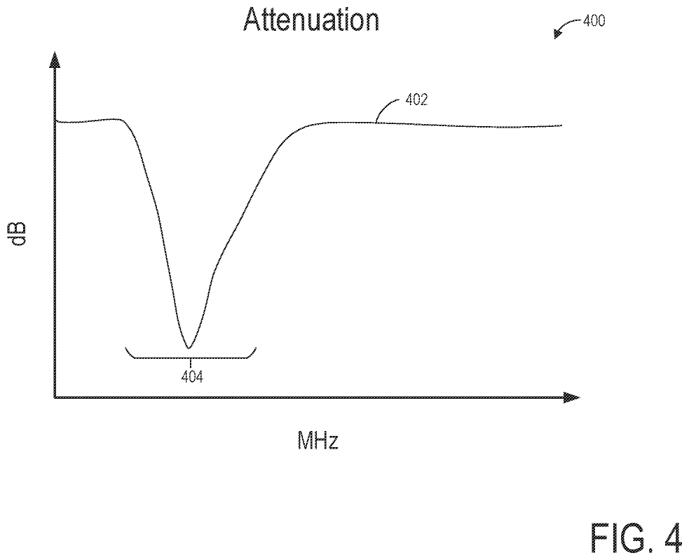

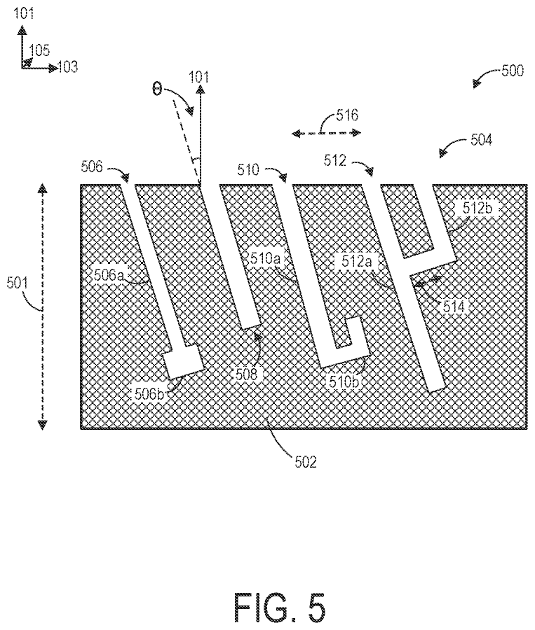

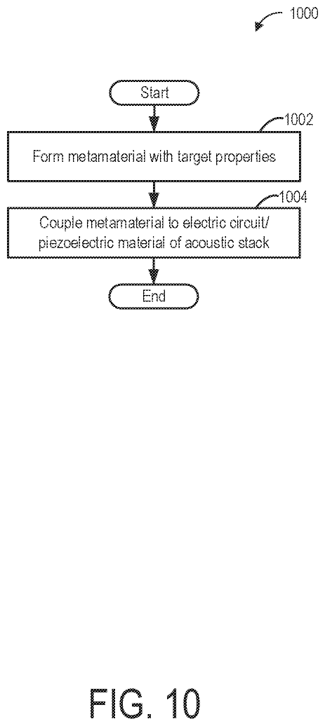

To illustrate an arrangement of acoustic components enabling use of a transducer for acoustic applications, an example of an acoustic stack including a modified dematching layer is depicted in FIG. 1A, coupled to a flex circuit, and FIG. 1B, coupled to at least one ASIC. Examples of absorption of acoustic energy by a conventional dematching layer and a modified dematching layer are shown in FIGS. 2 and 3, respectively. As described above, the modified dematching layer may be a metamaterial engineered to have a target band gap, as depicted in a graph plotting attenuation of acoustic energy by the metamaterial in FIG. 4. The modified dematching layer may be embedded with inclusions, examples of which are illustrated in FIGS. 5-9, that imparts the modified dematching layer with acoustic attenuating properties. An example of a method for fabricating a transducer with the modified dematching layer is shown in FIG. 10.

An ultrasound probe includes one or more active components for generating an ultrasonic signal. An example of an active component, or piezoelectric element 102 of an ultrasound probe is shown in schematic diagrams of an acoustic stack 100 in FIG. 1A, and 150 in FIG. 1B, having a central axis 104. A set of reference axes are provided, indicating a propagation (e.g., signal propagation) direction 101, an azimuth direction 103, and an elevation direction 105. In other examples, the set of reference axes may represent a z-axis 101, an x-axis 103, and a y-axis 105. The piezoelectric element 102 is shown in FIGS. 1A-1B with the central axis 104 parallel with the propagation direction or z-axis 101.

The piezoelectric element 102 may have a thickness, as defined along the propagation direction 101, that allows the piezoelectric element to generate an acoustic signal at a resonance frequency that is ¼ of a target operating frequency of the ultrasound probe. The ¼-λ piezoelectric element may offer increased sensitivity and bandwidth compared to a thicker ½-λ piezoelectric element.

It will be noted that while the acoustic stacks 100 and 150 are shown configured for a linear ultrasound probe and the propagation direction is described as parallel with the z-axis in FIGS. 1A-1B, other examples may include a propagation direction that is angled relative to the z-axis, depending on a shape of a piezoelectric element array. For example, the ultrasound probe may be curvilinear or phased array, thus generating non-linear beams that are not parallel with the z-axis. Furthermore, while the examples shown and described herein are directed to ultrasound applications, the methods and systems described below may be applicable to a variety of sensor array types.

While a single piezoelectric element is shown in FIGS. 1A-1B, the ultrasound probe may include a plurality of piezoelectric elements arranged in an array and individually coupled to an electrical energy source by wires. In some examples, the transducer may include an array of piezoelectric elements which may arranged in a variety of patterns, or matrices, including one-dimensional (1D) linear, two-dimensional (2D) square, 2D annular, etc. Each transducer may be electrically insulated from adjacent transducers but may all be coupled to common layers positioned above and below the piezoelectric element, with respect to the propagation direction 101. The plurality of piezoelectric elements and accompanying layers may be enclosed by an outer housing of the ultrasound probe which may be, for example, a plastic case with a variety of geometries. For example, the outer housing may be a rectangular block, a cylinder, or a shape configured to fit into a user's hand comfortably. As such, components shown in FIGS. 1A-1B may be adapted to have geometries and dimensions suitable to fit within the outer housing of the ultrasound probe.

The piezoelectric element 102 may be a block formed of a material, such as lead zirconate titanate, that deforms and vibrates when a voltage is applied by, for example, a transmitter. In some examples, the piezoelectric element 102 may be a single crystal with crystallographic axes, such as PMN-PT (Pb (Mg_{1/3}Nb_{2/3}) O₃—PbTiO₃). The vibration of the piezoelectric element 102 generates an ultrasonic signal formed of ultrasonic waves that are transmitted out of the ultrasound probe in a direction indicated by arrows 107, e.g., along the propagation direction 101. The piezoelectric element 102 may also receive ultrasonic waves, such as ultrasonic waves reflected from a target object, and convert the ultrasonic waves to a voltage. The voltage may be transmitted to a receiver of the ultrasound imaging system and processed into an image.

As illustrated in FIG. 1A, a flex circuit 112 may be coupled to the piezoelectric element 102. The flex circuit 112 may include electrodes 114 in direct contact with the piezoelectric element 102 to transmit the voltage via wires 115, the voltage converted from ultrasonic waves. The electrodes 114 may be coupled to a dielectric layer, such as polyimide. The wires 115 may be connected to a circuit board (not shown) to which a plurality of wires from electrodes of the plurality of piezoelectric elements may be fixed. The circuit board may be coupled to a coaxial cable providing electronic communication between the ultrasound probe and the receiver.

Alternatively, as shown in FIG. 1B, at least one ASIC 152 may be directly coupled to the piezoelectric element 102 and located below (e.g., behind) the piezoelectric element 102 relative to the z-axis 101. The ASIC 152 may convert the voltage from the piezoelectric element 102 into an electrical signal that is conveyed to a computing system 154 coupled to the ultrasound probe. The ASIC 152 may also supply a voltage for inducing vibration of the piezoelectric element 102. The ASIC 152 may therefore operate as a transmitter and/or a receiver.

As shown in FIGS. 1A-1B, an acoustic matching layer 120 may be arranged above the piezoelectric element 102, with respect to the propagation direction 101, oriented perpendicular to the central axis 104. The acoustic matching layer 120 may be a material positioned between the piezoelectric element 102 and a target object to be imaged. By arranging the acoustic matching layer 120 in between, the ultrasonic waves may first pass through the acoustic matching layer 120, and emerge from the acoustic matching layer 120 in phase, thereby reducing a likelihood of reflection at the target object. The acoustic matching layer 120 may shorten a pulse length of the ultrasonic signal, thereby increasing an axial resolution of the signal. In some examples, more than one acoustic matching layer may be included in the acoustic stack 100, 150.

The acoustic matching layer 120 may be positioned between a lens of the ultrasound probe and the piezoelectric element 102. The acoustic matching layer 120 may be composed of a material enabling high efficiency energy transfer, such as epoxy polyurethane, polystyrene, etc. An acoustic impedance gradient may be provided by the acoustic matching layer(s) 120 to allow the acoustic energy emitting from the ultrasound probe to penetrate tissue and be reflected back to the ultrasound probe from the tissue with minimal loss of signal.

In a conventional acoustic stack, a backing layer (not shown in FIGS. 1A-1B) may be arranged below (e.g., behind) the piezoelectric element, with respect to the propagation direction 101. The backing layer may be configured to absorb ultrasonic waves directed from the piezoelectric element 102 in a direction opposite of the direction indicated by arrows 107 and attenuate any stray ultrasonic waves deflected by the transducer and probe in directions other than directions useful for imaging, e.g., directions outside of a range of signal angles that may be transmitted and received by the ultrasound probe based on its specific size and frequency range. A bandwidth of the ultrasonic signal, as well as the axial resolution, may be increased by the backing layer.

An effect of the backing layer on axial resolution and bandwidth may be dependent on its thickness. For example, increasing the thickness of the backing layer may increase a capacity of the backing layer to attenuate, e.g., dampen, scattered and stray acoustic energy. Thus, in instances where reducing the size of the ultrasound probe is desirable, decreasing the thickness of the backing layer may be constrained by degradation of the ultrasound probe performance.

In the examples of FIGS. 1A-1B, however, the backing layer may be omitted. Instead, the acoustic stack may be configured with a modified dematching layer 126. In other examples, however, the backing layer may be included but may have a reduced thickness compared to backing layers in conventional acoustic stacks. The modified dematching layer 126 may be formed of a metamaterial having a matrix with an acoustic impedance that is higher than that of the piezoelectric element 102. For example, the matrix may be formed of tungsten carbide. The matrix of the modified dematching layer 126 may therefore be referred to as a high acoustic impedance material. The matrix may further be embedded with inclusions having an acoustic impedance that is similar to that of the piezoelectric element 102. The inclusions may therefore be formed of and referred to as a low acoustic impedance material.

The inclusions embedded in the matrix of the modified dematching layer 126 may be cavities, bubbles of a fluid, or particles. In this way, an internal structure of the modified dematching layer 126 may be moderated, during fabrication, to achieve target acoustic impedance and acoustic attenuation characteristics. By combining both high and low acoustic impedance materials into the modified dematching layer 126, the modified dematching layer 126 may operate as both a dematching layer and as a backing (e.g., backing layer). For example, the matrix of the modified dematching layer 126 may reflect a majority of the ultrasonic signal received by the ultrasound probe out of a front of the ultrasound probe (e.g., along the propagation direction 101), allowing a reflected portion of the ultrasonic signal to be used for imaging. The inclusions embedded in the matrix may demonstrate acoustic attenuation in addition to low acoustic impedance, and may absorb scattered, stray acoustic energy travelling through the modified dematching layer 126. Further details of the modified dematching layer 126 are provided further below.

The modified dematching layer, e.g., the modified dematching layer 126 of FIGS. 1A-1B, may have properties of a conventional dematching layer but may additionally absorb and attenuate acoustic energy travelling in directions other than a signal propagation direction, as described above. Acoustic characteristics of the modified dematching layer may be tuned to include effects of a backing by employing a metamaterial as the modified dematching layer.

For example, a simplified diagram of a conventional acoustic stack 200 for a transducer is depicted in FIG. 2 to provide a comparison to a modified acoustic stack 300 illustrated in FIG. 3. The conventional acoustic stack 200 includes a piezoelectric material 202 and a dematching layer 204. A backing may be included in the conventional acoustic stack 200 but not shown in FIG. 2 for illustrative purposes. A direction of acoustic signal transmission, e.g., signal propagation along the z-axis 101, is indicated by arrow 206 and scattering and/or reflection of the acoustic signal through the dematching layer 204 is indicated by arrows 208.

At the dematching layer 204, backscattered acoustic energy, as indicated by a first arrow 208a of the arrows 208, may travel from the piezoelectric material 202 to a rear surface 210 of the dematching layer 204 in a direction opposite of signal propagation. The acoustic energy may be reflected at the rear surface 210, as indicated by a second arrow 208b of the arrows 208, and directed to an interface between the piezoelectric material 202 and the dematching layer 204, as indicated by a third arrow 208c of the arrows 208, where the acoustic energy may again be reflected. The acoustic energy is not attenuated by the dematching layer 204 and an intensity of the reflected acoustic energy is therefore not decreased while travelling through the dematching layer 204. As such, the scattered acoustic energy may reverberate through the dematching layer 204 and interfere with signal reception at the piezoelectric material 202.

The backing, positioned below the dematching layer 204, may attenuate at least some of the acoustic energy that is directed thereto, e.g., along the direction indicated by the first arrow 208a. However, in some examples, a portion of the acoustic energy travelling towards the backing may be deflected at an interface between the dematching layer 204 and the backing, which may be directed away from the backing, as indicated by the second and third arrows 208b, 208c. The deflected portion is not attenuated by the dematching layer, as described above. Furthermore, a thickness of the backing may be greater than either of the piezoelectric material 202 or the dematching layer 204 in order for the backing to effectively attenuate the scattered acoustic energy.

In contrast, as shown in FIG. 3 in another simplified diagram of a modified acoustic stack 300, when a piezoelectric material 302 is paired with a modified dematching layer 304, as described herein, attenuation of scattered and reflected acoustic energy is enabled. For example, an effect of the modified dematching layer 304 on acoustic energy scattering and reflection is illustrated in FIG. 3. Transmission of an acoustic signal from the piezoelectric material 302 is indicated by arrow 306 and scattering of acoustic energy is indicated by arrows 308.

The scattered acoustic energy may travel to a rear surface 310 of the modified dematching layer 304, as indicated by a first arrow 308a of the arrows 308. The acoustic energy may be reflected at the rear surface 310, as indicated by a second arrow 308b of the arrows, and travel to an interface between the piezoelectric material 302 and the modified dematching layer 304, as indicated by a third arrow 308c of the arrows 308.

As the acoustic energy passes through the modified dematching layer 304, the acoustic energy may interact with inclusions embedded therein, the inclusions having low acoustic impedance and high acoustic attenuation. Further, the acoustic energy may be reflected by the inclusions, thereby increasing an amount of time that the acoustic energy travels through the dematching layer 304 and increasing a number of interactions between the acoustic energy and the inclusions. The acoustic energy may thereby be dampened as it travels through the dematching layer 304, as shown by arrows 308, which may suppress reverberation of the acoustic energy and increase a performance of the transducer relative to the transducer with the conventional dematching layer 204 of FIG. 2.

By enabling the modified dematching layer 304 to attenuate scattered acoustic energy passing therethrough, a greater portion of the scattered acoustic energy may be absorbed and attenuated, compared to the conventional acoustic stack 200. For example, by the time the scattered acoustic energy reaches a rear side of the modified dematching layer 304, e.g., a side distal to the piezoelectric material 302, the acoustic energy may be reduced in intensity. As a result, less acoustic energy may enter the backing, when present, which may be arranged below the modified dematching layer 304, and the thickness of the backing may be decreased accordingly. In some examples, the modified dematching layer 304 may be able to attenuate the scattered acoustic energy to an extent where the backing may be obviated.

A metamaterial used as a modified dematching layer for an acoustic stack may be an acoustic metamaterial, which may be an artificially structured material with inclusions incorporated into a matrix of the metamaterial in a controlled manner. In some examples, the inclusions may be periodically distributed, such as when the acoustic metamaterial is a phononic crystal, which may, for example, drive Bragg scattering that creates destructive interferences for specific angles. In other examples, the inclusions may be distributed in a non-uniform pattern, such as when the inclusions are configured for Fabry-Perot resonances. The metamaterial may be fabricated by various known techniques, including but not limited to machining, microfabrication, molding, casting, wet-chemical techniques, in addition to 3D printing and lithographic techniques, as described above. Further, the metamaterial may be tuned to have a target acoustic band gap, unlike composite materials, that corresponds to a resonance frequency of the piezoelectric material. This allows the modified dematching layer to effectively attenuate frequencies at or within a threshold range of the resonance frequency of the piezoelectric material.

For example, as shown in graph 400 in FIG. 4, plot 402 represents attenuation of acoustic energy, in decibels, relative to frequency, in megahertz, for a metamaterial. The metamaterial has a band gap (e.g., an acoustic band gap) indicated by bracket 404, which may encompass a resonance frequency of a piezoelectric material to which the metamaterial is to be coupled. As described above, the band gap may vary depending on a structure of the metamaterial. The band gap of the metamaterial may result from destructive interference of a resonance frequency of the inclusions with the resonance frequency of the piezoelectric material. As an example, a frequency range of the transducer may be 2.0 to 10.0 MHz and the band gap of the metamaterial may be 4 to 6 MHz. By adjusting parameters such as bulk modulus, mass density, and chirality, the metamaterial may be tuned to have desired acoustic properties for transmitting, trapping, and/or amplifying acoustic waves.

In some examples, the metamaterial may be engineered with a target difference in acoustic impedance between the high impedance material and the low impedance material. For example, the high impedance material may be cobalt-bonded WC having an acoustic impedance of approximately 100 MR and the low impedance materials may be silicon rubber having a much lower acoustic impedance of approximately 1.3 MR. In this way, the low impedance material, which may also be a high acoustic attenuating material, may not adversely affect an overall acoustic impedance of the modified dematching layer.

The metamaterial may therefore be configured to maintain a desired acoustic impedance in spite of a presence of the inclusions, which have a lower acoustic impedance. For example, the metamaterial may be engineered to have an acoustic impedance ratio relative to the piezoelectric material of at least 2. As one example, the metamaterial may have an acoustic impedance as low as 70 MR when coupled to a PZT having an acoustic impedance of 35 MR.

Absorption and attenuation of acoustic energy by the metamaterial is enabled based on a density and distribution of inclusions, rather than a particular material from which the inclusions may be formed. The inclusions may exhibit Fabry-Perot, Minnaert, or Helmholtz resonances, as examples, when activated by vibration at the piezoelectric material, and the inclusions may absorb the acoustic energy scattered into the metamaterial via destructive interferences. For example, the inclusions may absorb the acoustic energy at the acoustic band gap of the metamaterial.

The inclusions may have a variety of shapes and be formed from various materials. For example, the inclusions may be shaped as pillars, spheres, plates, or have a honey-comb pattern. Materials for the inclusions may include metals, polymers, air, gases, liquids, etc. In some examples, the inclusions may be particles that are coated or uncoated. In other examples, the inclusions may be cavities or trapped bubbles of air or a fluid. As described above, the inclusions may be distributed according to a target application and effect on acoustic energy. A distribution of the inclusions within a high acoustic impedance matrix of the metamaterial may be uniform, e.g., having periodicity, or nonuniform. For example, when non-uniform, the inclusions may be concentrated in a particular region of the matrix to leverage Fabry-Perot resonances.

A first example of a metamaterial 500 which may be included in an acoustic stack of a transducer having a ¼-λ resonance frequency is depicted in FIG. 5. The metamaterial 500 may have a matrix 502 with a high acoustic impedance and one or more pillar-shaped inclusions 504 of lower acoustic impedance. The matrix 502 may be, as one example, formed of WC and may have a density of 15 g/cm³. The pillar-shaped inclusions 504 may be cavities that extend into at least a portion of a thickness 501 of the metamaterial 500 and may have cross-sectional areas of various geometries, such as square, rectangular, circular, elliptical, etc. For example, the pillar-shaped inclusions 504 may extend along 10-90% of the thickness 501 of the metamaterial 500. The pillar-shaped inclusions 504 may extend downwards, relative to the z-axis 101, from an upper surface of the metamaterial 500 that is configured to be in contact with a piezoelectric material of the acoustic stack. The one or more pillar-shaped inclusions 504 may be of a same type or may be of different types, as exemplified by examples of the pillar-shaped inclusions 504 illustrated in FIG. 5.

The pillar-shaped inclusions 504 includes a first pillar-shaped inclusion 506 which may have a neck 506a linked to an end cavity 506b. A geometry of the first pillar-shaped inclusion 506 may allow the first pillar-shaped inclusion to act as a Helmhotz resonator. A frequency of the first pillar-shaped inclusion 506 as the Helmholtz resonator may depend on dimensions (such as length and width when the first pillar-shaped inclusion 506 has a square cross-sectional area) of the neck 506a relative to dimensions of the end cavity 506b (such as length, width, and depth). The cross-sectional shape of the pillar-shaped inclusions 504 may also affect their capacity for attenuating acoustic energy. For example, when fabricating the metamaterial 500, the dimensions and shape of the first pillar-shaped inclusion 506 may be selected such that the frequency of acoustic energy generated at the first pillar-shaped inclusion may match a resonance frequency of the piezoelectric material.

A second pillar-shaped inclusion 508 may be shaped as a pillar extending linearly into the matrix 502. Dimensions of the second pillar-shaped inclusion 508 may be configured to enable the second pillar-shaped inclusion 508 to interact destructively with resonance frequencies of the piezoelectric material. The second pillar-shaped inclusion 508, as well as a third pillar-shaped inclusion 510, may similarly have a variable cross-sectional geometry, as described above. The pillar-shaped inclusions may also include the third pillar-shaped inclusion 510 having a hooked shape, which may be a Helmholtz resonator similar to the first pillar-shaped inclusion 506. Relative dimensions of a neck 510a and an end cavity 510b of the third pillar-shaped inclusion 510 may control resonance frequencies generated by the third pillar-shaped inclusion 510.

The pillar-shaped inclusions 504 may further include a fourth pillar-shaped inclusion 512 which may be formed or more than one interconnected pillars. For example, the fourth pillar-shaped inclusion 512 may have a first interconnected pillar 512a and a second interconnected pillar 512b, which are arranged parallel with one another and have different lengths. A frequency of acoustic energy generated at the fourth pillar-shaped inclusion 512 may be varied based on, for example, a distance 514 between the interconnected pillars of the fourth pillar-shaped inclusion 512, as well as on relative lengths of the interconnected pillars.

An effect of the pillar-shaped inclusions 504 on attenuating acoustic energy may also be optimized based on a distance 516 between adjacent pillar-shaped inclusions and on an angle θ that the pillar-shaped inclusions 504 extend through the metamaterial 500. For example, the angle θ may be any angle between −80 to +80 relative to the z-axis 101. The pillar-shaped inclusions may therefore be incorporated into the metamaterial 500 according to a variety of parameters, e.g., length, width, end cavity size relative to neck size, spacing, angle, mixture of inclusion types, etc., to obtain a metamaterial with a target band gap.

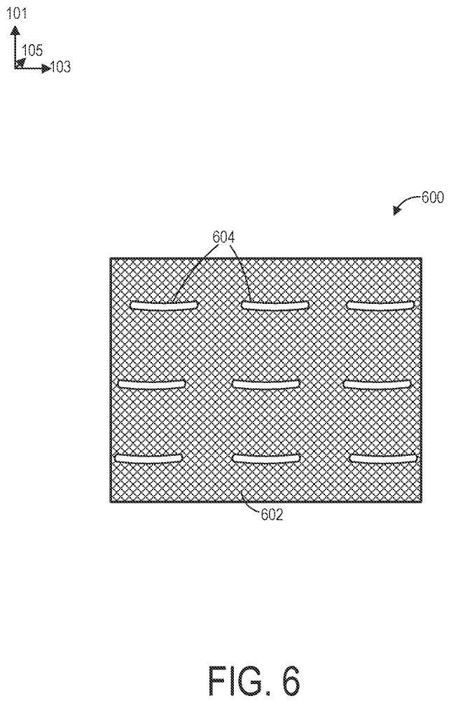

As shown in FIG. 6 in a second example of a metamaterial 600 that may be included in an acoustic stack of a transducer having a ¼-λ resonance frequency, the metamaterial 600 may have a matrix 602 with high acoustic impedance, as described above, and inclusions 604 that are disc or plate-shaped. The inclusions 604 may be cavities having low acoustic impedance and providing high acoustic attenuation.

Although the inclusions 604 are shown in FIG. 6 arranged in columns and spaced evenly apart, the inclusions 604 may be distributed differently in other examples. For example, the inclusions 604 may be positioned randomly and not in columns, may be concentrated in one or more regions of the metamaterial 600, may have different dimensions than shown in FIG. 6 and/or from one another, and/or may be oriented differently within the metamaterial 600. As an example, the inclusions 604 may be oriented perpendicular to the x-axis 103 rather than the z-axis 101, or to the y-axis 105, or may be angled relative to any of the axes. As described above, an arrangement of the inclusions 604 within the metamaterial 600 may be selected based on application and target acoustic properties.

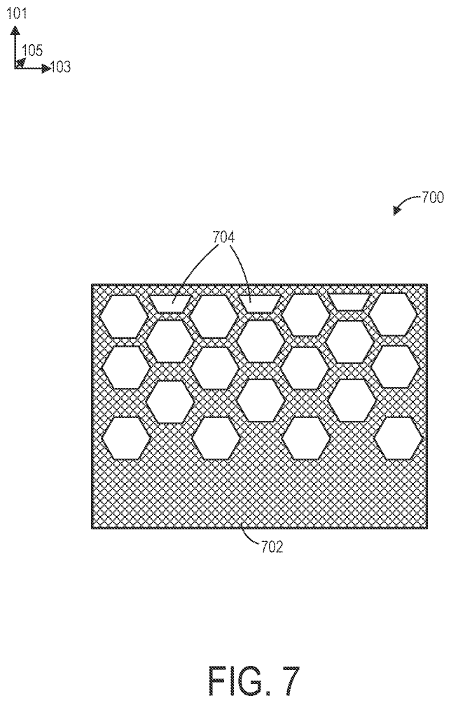

A third example of a metamaterial 700 for an acoustic stack of a transducer having a ¼-λ resonance frequency is illustrated in FIG. 7. A matrix 702 of the metamaterial 700, the matrix 702 being formed of a material with high acoustic impedance, may have inclusions 704 that are also cavities. The inclusions 704 may be hexagonal, as an example, forming a honey-comb pattern. The honey-comb pattern may be periodic, with the inclusions 704 having a uniform geometry and spaced evenly apart, or may have irregular, e.g., varied, geometry and spacing, or any combination thereof. Further, the inclusions 704 may be distributed, with or without periodicity, throughout the metamaterial 700 or concentrated in one or more regions of the metamaterial 700. Additionally, the inclusions 704 may be of variable sizes.

An orientation of the inclusions 704 may also vary from that shown in FIG. 7. For example, the inclusions 704 may extend along the y-axis 105 as shown FIG. 7, along the x-axis 103, or along the z-axis 101. In addition, the inclusions 704 may extend entirely through the metamaterial 700 or through only a portion thereof, along a given axis.

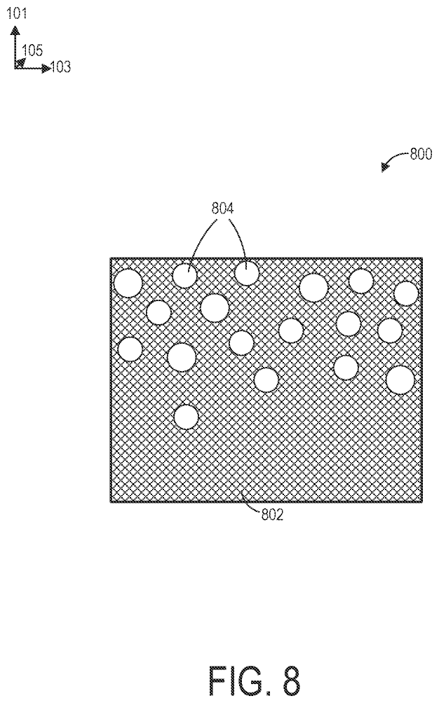

A fourth example of a metamaterial 800 for an acoustic stack of a transducer having a ¼-λ resonance frequency is illustrated in FIG. 8. The metamaterial 800, similar to the previous examples, may have a matrix 802 with high acoustic impedance and inclusions 804 with low acoustic impedance but high acoustic attenuation. The inclusions 804 may be bubbles of air, or of another fluid, trapped within the matrix 802 of the metamaterial 800.

The inclusions 804 may be uniformly spaced apart, or may have a relatively random spacing within the matrix 802 but may be concentrated in at least one region, as shown in FIG. 8, or may be distributed throughout the metamaterial 800. A number and size of the inclusions 804 may be adjusted according to target acoustic properties to be provided by the metamaterial 800.

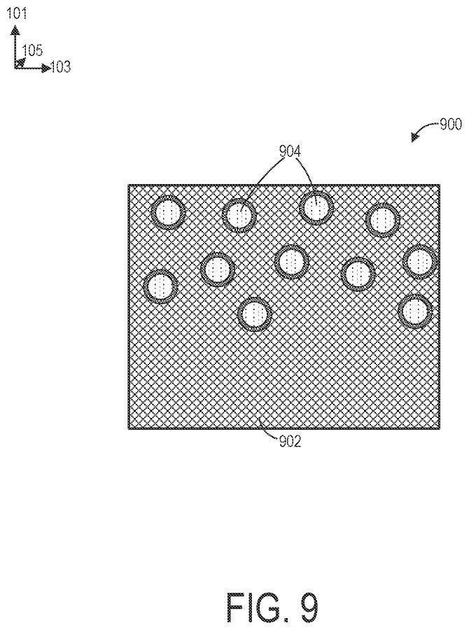

A fifth example of a metamaterial 900 which may be included in an acoustic stack of a transducer having a ¼-λ resonance frequency is depicted in FIG. 9. The metamaterial 900 may have a matrix 902 having high acoustic impedance, as described above, and inclusions 904 shaped as spheres. The inclusions 904 may be particles formed of a material with low acoustic impedance and may be distributed within the matrix 902 to deliver high acoustic attenuation. As shown in FIG. 9, the particles may be coated but may be uncoated in other examples.

In one example, the inclusions 904 may be polymer-coated particles of stainless steel. Other materials able to yield desired acoustic properties have been contemplated, however. In addition, the inclusions 904 may have other geometries besides spherical.

A diameter of the inclusions 904, a coating thickness, a spacing between the inclusions 904, a density, and a distribution of the inclusions 904 within the matrix 902 may vary according to a target band gap of the metamaterial 900. Furthermore, while the inclusions 904 are illustrated having uniform diameters, in other examples, the inclusions 904 may have different diameters (e.g., sizes) within a given metamaterial.

It will be appreciated that the examples shown in FIGS. 5-9 are non-limiting examples of metamaterials that may be incorporated into an acoustic stack as a modified dematching layer. Numerous variations in the configurations shown and described are possible without departing from the scope of the present disclosure. Further, combinations of the different types of inclusions are possible within a given modified dematching layer.

Regardless of the particular metamaterial configuration used, the dematching layer may provide desirable attributes of an effective dematching layer for tuning a resonance frequency of a transducer with a ¼-λ piezoelectric material and also absorbing scattered and reflecting acoustic energy. By forming the modified dematching layer from the metamaterial, the modified dematching layer may be fabricated to deliver specific, targeted acoustic effects. For example, by selecting a type, distribution, and geometry of inclusions, the modified dematching layer may have an acoustic band gap matching a resonance frequency of the piezoelectric material. The modified dematching layer may rely on destructive interference between the resonance frequency of the piezoelectric material and Fabry-Perot, Minnaert, or Helmholtz resonances of the inclusions to absorb/attenuate acoustic energy scattered into the modified dematching layer. As a result, a backing may be omitted from the acoustic stack or may at least be reduced in thickness.

In examples where a flex circuit is coupled to the acoustic stack, e.g., as shown in FIG. 1A, the acoustic properties of the modified dematching layer may also be tuned to attenuate natural resonance frequencies of the flex circuit material, thereby reducing acoustic interferences imposed by the flex circuit. In examples where one or more ASICs are coupled to the acoustic stack, e.g., as shown in FIG. 1B, use of the modified dematching layer may obviate incorporation of a backing between the one or more ASICs and the modified dematching layer. A demand for a backing fabricated with electrical connections extending therethrough may be precluded, which may simplify manufacturing of the acoustic stack and reduce costs. Overall, a resolution of the transducer may be enhanced while a footprint of the transducer may be decreased.

A method 1000 for forming an acoustic stack with a modified dematching layer is depicted in FIG. 10. The modified dematching layer may be any of the examples of the modified dematching layer, or any combination thereof, shown and described above with reference to FIGS. 5-9. Method 1000 may be executed by one or more of operators, and machines and equipment suitable for carrying out steps included therein.

At 1002, method 1000 includes forming a metamaterial having target acoustic properties. For example, the metamaterial may include a matrix comprising a high acoustic impedance material, such as tungsten carbide. The metamaterial may be fabricated via any known technique, e.g., 3D printing, machining, microfabrication, molding, casting, wet-chemical techniques, etc., to embed the matrix with inclusions formed of a material that is different than the matrix. More specifically, the inclusions may be formed of a material with low acoustic impedance.

Characteristics of the inclusions, such as distribution, density, type (e.g., cavities vs. particles, coated particles vs. uncoated, material of the particles, fluid occupying the cavities), and geometry of the inclusions may be determined based on a resulting effect on absorption and attenuation of stray acoustic energy. For example, the characteristics may be selected to achieve a metamaterial with an acoustic band gap that encompasses a resonance frequency of a piezoelectric material to which the modified dematching layer is to be coupled.

At 1004, method 1000 includes coupling the modified dematching layer, formed of the metamaterial, to other components of the acoustic stack. For example, the modified dematching layer may be placed in face-sharing contact with the piezoelectric material at a first face of the modified dematching layer and in face-sharing contact with one or more ASICs at a second face of the modified dematching layer, the first face opposite of the second face. A matching layer may be coupled to the piezoelectric material at a side of the piezoelectric material opposite of the modified dematching layer. The resulting acoustic stack may be similar to the acoustic stack 150 of FIG. 1B. As another example, the modified dematching layer may be coupled to the piezoelectric material with an electrode of a flex circuit positioned therebetween. The piezoelectric material may also be coupled to a matching layer arranged opposite of the modified dematching layer. The resulting acoustic stack may be similar to the acoustic stack 100 of FIG. 1A.

Further, in some examples, a backing may be included. When present, the backing may be located between the modified dematching layer and the piezoelectric material. The backing may have a thickness that is reduced relative to backings included in acoustic stacks utilizing conventional dematching layers (e.g., materials that are not metamaterials optimized for acoustic probe applications). The acoustic stack may undergo processing, such as lamination, to promote adherence of the components to one another.

FIGS. 1A-9 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The disclosure also provides support for a modified dematching layer for a transducer, the modified dematching layer comprising: a matrix having a first acoustic impedance, and inclusions embedded in the matrix, the inclusions having a second acoustic impedance lower than the first acoustic impedance and configured to attenuate acoustic energy at an acoustic band gap of the modified dematching layer. In a first example of the system, the modified dematching layer is formed of a metamaterial, and wherein the acoustic band gap of the metamaterial corresponds to a resonance frequency of a piezoelectric material. In a second example of the system, optionally including the first example, the modified dematching layer is included in an acoustic stack without a backing positioned between the modified dematching layer and a piezoelectric material. In a third example of the system, optionally including one or both of the first and second examples, the modified dematching layer is included in an acoustic stack with the modified dematching layer in face-sharing contact with one or more ASICs arranged between the modified dematching layer and a piezoelectric material. In a fourth example of the system, optionally including one or more or each of the first through third examples, the modified dematching layer is included in a first acoustic stack with a first backing, and wherein the first backing has a reduced thickness relative to a second backing that is included in a second acoustic stack having a dematching layer without the inclusions. In a fifth example of the system, optionally including one or more or each of the first through fourth examples, the inclusions are one or more of embedded particles, cavities, and fluid bubbles. In a sixth example of the system, optionally including one or more or each of the first through fifth examples, the inclusions are distributed within the matrix to destructively interfere with resonance frequencies of a piezoelectric material based on one or more of Fabry-Perot. Minnaert. and Helmholtz resonances at the inclusions. In a seventh example of the system, optionally including one or more or each of the first through sixth examples, the inclusions are concentrated at one or more regions of the matrix. In a eighth example of the system, optionally including one or more or each of the first through seventh examples, the inclusions are distributed periodically through the matrix, and wherein the modified dematching layer is a phononic crystal.

The disclosure also provides support for a method for a fabricating a transducer, comprising: fabricating a metamaterial with an acoustic band gap corresponding to a resonance frequency of a piczoelectric material, the metamaterial having a matrix with a first acoustic impedance and inclusions with a second acoustic impedance, the second acoustic impedance lower than the first acoustic impedance, and incorporating the metamaterial into an acoustic stack, wherein the metamaterial is both a dematching layer and a backing of the acoustic stack. In a first example of the method, the piezoelectric material has a ¼-wavelength resonance frequency, and wherein the metamaterial is arranged in the acoustic stack with an electrical circuit positioned between the metamaterial and the piezoelectric material and in direct contact with each of the metamaterial and the piezoelectric material. In a second example of the method, optionally including the first example, the acoustic band gap also corresponds to a resonance frequency of a flex circuit coupled to the acoustic stack. In a third example of the method, optionally including one or both of the first and second examples, acoustic energy generated by the piezoelectric material is attenuated by the inclusions of the metamaterial. In a fourth example of the method, optionally including one or more or each of the first through third examples, the inclusions are pillar-shaped cavities extending into the metamaterial from an upper surface of the metamaterial, and wherein attenuation of acoustic energy by the inclusions is optimized based on one or more of a depth of the inclusions into a thickness of the metamaterial, a spacing between the inclusions, a geometry of the inclusions, and an angle of the inclusions relative to a direction of signal propagation.

The disclosure also provides support for an acoustic stack, comprising: a piezoelectric material, an electrical circuit in contact with the piezoelectric material, and a modified dematching layer positioned adjacent to the piezoelectric material with the electrical circuit positioned therebetween, the modified dematching layer formed of a metamaterial having a matrix with a first, higher acoustic impedance and inclusions with a second, lower acoustic impedance, and wherein the inclusions are distributed within the matrix to attenuate acoustic energy at a resonance frequency of the piezoelectric material. In a first example of the system, the metamaterial has an acoustic impedance that is at least two times an acoustic impedance of the piezoelectric material. In a second example of the system, optionally including the first example, the inclusions are cavities shaped as one or more of pillars, discs, and a honey-comb pattern. In a third example of the system, optionally including one or both of the first and second examples, the inclusions are one or more of coated and uncoated particles. In a fourth example of the system, optionally including one or more or each of the first through third examples, the inclusions are bubbles of a fluid. In a fifth example of the system, optionally including one or more or each of the first through fourth examples, the inclusions resonate at frequencies that destructively interfere with stray acoustic energy travelling through the metamaterial.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

The invention claimed is:

1. A modified dematching layer for a transducer, the modified dematching layer comprising:
a matrix having a first acoustic impedance; and
inclusions embedded in the matrix, the inclusions having a second acoustic impedance lower than the first acoustic impedance and configured to attenuate acoustic energy at an acoustic band gap of the modified dematching layer.

2. The modified dematching layer of claim 1, wherein the modified dematching layer is formed of a metamaterial, and wherein the acoustic band gap of the metamaterial corresponds to a resonance frequency of a piezoelectric material.

3. The modified dematching layer of claim 1, wherein the modified dematching layer is included in an acoustic stack without a backing positioned between the modified dematching layer and a piezoelectric material.

4. The modified dematching layer of claim 1, wherein the modified dematching layer is included in an acoustic stack with the modified dematching layer in face-sharing contact with one or more ASICs arranged between the modified dematching layer and a piezoelectric material.

5. The modified dematching layer of claim 1, wherein the modified dematching layer is included in a first acoustic stack with a first backing, and wherein the first backing has a reduced thickness relative to a second backing that is included in a second acoustic stack having a dematching layer without the inclusions.

6. The modified dematching layer of claim 1, wherein the inclusions are one or more of embedded particles, cavities, and fluid bubbles.

7. The modified dematching layer of claim 1, wherein the inclusions are distributed within the matrix to destructively interfere with resonance frequencies of a piezoelectric material based on one or more of Fabry-Perot, Minnaert, and Helmholtz resonances at the inclusions.

8. The modified dematching layer of claim 1, wherein the inclusions are concentrated at one or more regions of the matrix.

9. The modified dematching layer of claim 1, wherein the inclusions are distributed periodically through the matrix, and wherein the modified dematching layer is a phononic crystal.

10. A method for a fabricating a transducer, comprising:
fabricating a metamaterial with an acoustic band gap corresponding to a resonance frequency of a piezoelectric material, the metamaterial having a matrix with a first acoustic impedance and inclusions with a second acoustic impedance, the second acoustic impedance lower than the first acoustic impedance; and
incorporating the metamaterial into an acoustic stack, wherein the metamaterial is both a dematching layer and a backing of the acoustic stack.

11. The method of claim 10, wherein the piezoelectric material has a ¼-wavelength resonance frequency, and wherein the metamaterial is arranged in the acoustic stack with an electrical circuit positioned between the metamaterial and the piezoelectric material and in direct contact with each of the metamaterial and the piezoelectric material.

12. The method of claim 10, wherein the acoustic band gap also corresponds to a resonance frequency of a flex circuit coupled to the acoustic stack.

13. The method of claim 10, wherein acoustic energy generated by the piezoelectric material is attenuated by the inclusions of the metamaterial.

14. The method of claim 10, wherein the inclusions are pillar-shaped cavities extending into the metamaterial from an upper surface of the metamaterial, and wherein attenuation of acoustic energy by the inclusions is optimized based on one or more of a depth of the inclusions into a thickness of the metamaterial, a spacing between the inclusions, a geometry of the inclusions, and an angle of the inclusions relative to a direction of signal propagation.

15. An acoustic stack, comprising:
a piezoelectric material;
an electrical circuit in contact with the piezoelectric material; and
a modified dematching layer positioned adjacent to the piezoelectric material with the electrical circuit positioned therebetween, the modified dematching layer formed of a metamaterial having a matrix with a first, higher acoustic impedance and inclusions with a second, lower acoustic impedance, and wherein the inclusions are distributed within the matrix to attenuate acoustic energy at a resonance frequency of the piezoelectric material.

16. The acoustic stack of claim 15, wherein the metamaterial has an acoustic impedance that is at least two times an acoustic impedance of the piezoelectric material.

17. The acoustic stack of claim 15, wherein the inclusions are cavities shaped as one or more of pillars, discs, and a honey-comb pattern.

18. The acoustic stack of claim 15, wherein the inclusions are one or more of coated and uncoated particles.

19. The acoustic stack of claim 15, wherein the inclusions are bubbles of a fluid.

20. The acoustic stack of claim 15, wherein the inclusions resonate at frequencies that destructively interfere with stray acoustic energy travelling through the metamaterial.
